(19) United States
(12) Patent Application Publication
(10) Pub. No.: US 2009/0264796 A1
(43) Pub. Date: Oct. 22, 2009

(54) Virtual reality system for rehabilitation of low back pain

(75) Inventors: Malcolm Pope, Kowloon (CN); Daniel Hung Kay Chow, Kowloon (CN)

Correspondence Address: MALCOLM POPE, THE HONG KONG POLYTECHNIC UNIVERSITY, HUNG HOM, KOWLOON, HONG KONG, CN

(73) Assignee: The Hong Kong Polytechnic University

(21) Appl. No.: 12/081,729

(22) Filed: Apr. 21, 2008

(51) Int. Cl. A61B 5/11; A61B 5/107

(52) U.S. Cl. 600/594; 600/595

(57) ABSTRACT

The present invention relates to a virtual reality feedback system to measure trunk orientation of a low back pain patient, and then displaying the measurement information on a display device for the benefit of the patient.

### BACKGROUND

Low Back Pain (LBP) is highly prevalent worldwide, with around 80% of individuals suffering from LBP at some time in their lives. Chronic low back pain (CLBP) is defined as when symptoms exceed 6 months. This is extremely costly. Several studies have reported that less than half the individuals disabled by LBP for more than 6 months return to work, and that by 2 years return to work is virtually 0% (Clinical Standards Advisory Group 1994). The development of CLBP is usually defined by time period. Most episodes of LBP resolve within 4-12 weeks, but if LBP persists beyond 6 months it should then be classified as chronic (Waddell 1996).

Biofeedback is the use of instrumentation to make patients aware of physiological processes (Basmajian, 1989; Dursun 2004, Moreland and Thomson 1994). The field has been nicely summarized by Huang et al (2006). Paraspinal electromyographic (EMG) biofeedback has been used during static and dynamic movement training of CLBP patients but with mixed results (Hasenbring et al, 1999; Newtonjohn et al, 1995). Learning appropriate muscle activity and postural control during dynamic conditions would be an advantage in the treatment and prognosis of CLBP. However, “appropriate” postural behaviour remains unclear. Classically, it has been thought that any decrease in the EMG activity during motor activity work may be an advantage. However, it is likely that both very fast and very slow responses put a person at an increased risk of overloading musculoskeletal structures. Our previous results showed that the magnitude of the muscle responses to sudden load was lower in CLBP patients than in healthy participants (Magnusson et al, 1998).

Biofeedback provides patients with sensorimotor impairments with a tool to regain the ability to better assess different physiological responses and to relearn control of those responses (Hilgard and Bower, 1975). Biofeedback aims to improve a patient's motor control by providing visual or audio feedback of such signals as EMG positional or force parameters in real time (Fernando and Basmajian, 1978; Wolf, 1983). The physiological feedback signals have included EMG (Hasenbring et al, 1999; Newtonjohn et al, 1995; Bush et al, 1985; Jones and Wolf, 1980; Nouwen and Solinger, 1979; Wolf et al, 1989), joint angle (Koheil et al, 1980; Colborne et al, 1993; Dursun et al, 1996), position (Hock, 2000; Conatell et al, 2005; Ekes et al, 1995; Montoya et al, 1994; Aruin et al, 2000), and pressure or ground reaction force (Winstein et al, 1989, Ariun et al, 2000; Simmons, 1998).

It is an object of the present invention to utilize biofeedback to aid in the treatment of low back pain, and chronic back pain.

### DESCRIPTION

The present invention relates to a virtual reality feedback system to measure trunk orientation of a low back pain patient, and then displaying the measurement information on a display device for the benefit of the patient.

These and other features, aspects, and advantages of the apparatus and methods of the present invention will become better understood from the following description, appended claims, and accompanying drawings where:

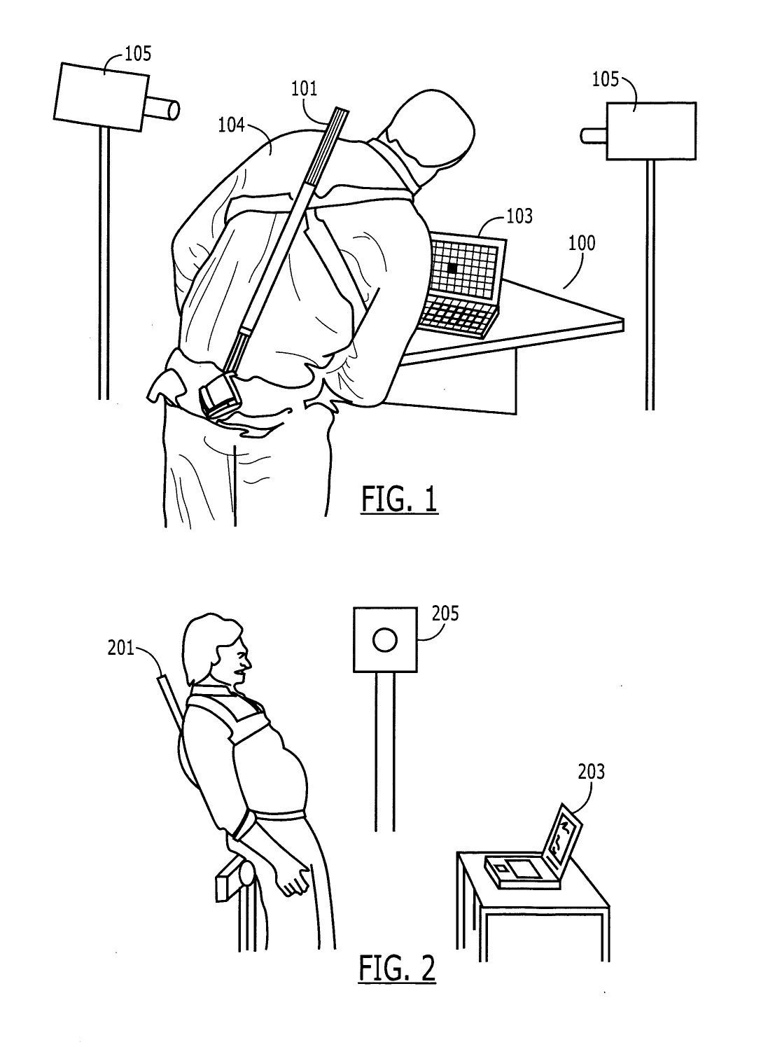

FIG. 1 is a backside view of the present invention;

FIG. 2 is a side view of the present invention;

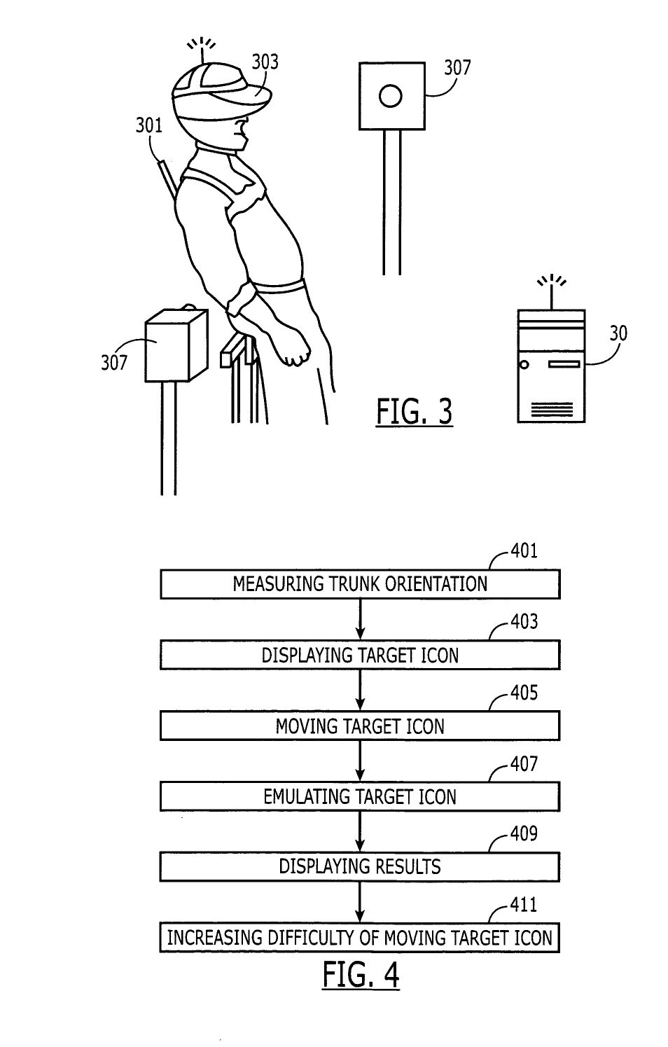

FIG. 3 is an embodiment of the present invention wherein the display is a head-mounted display;

FIG. 4 is a method of utilizing the system of the present invention;

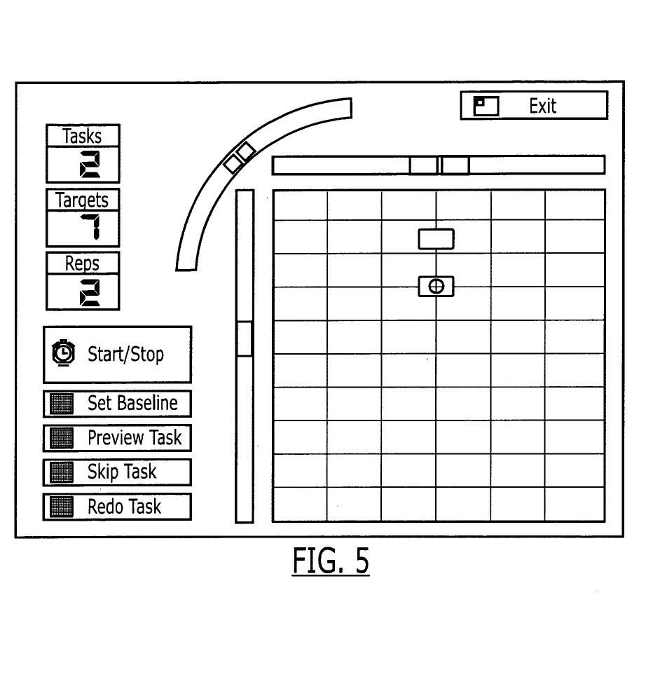

FIG. 5 is an example of the feedback received by a user.

The following description of certain exemplary embodiment(s) is merely exemplary in nature and is in no way intended to limit the invention, its application, or uses. Throughout this description, the term “chronic back pain” shall refer to back pain lasting longer than 6 months.

Now, to FIGS. 1-5,

FIG. 1 is an embodiment of the system 100 of the present invention. The system 100 includes a posture and joint deviation device 101 and a display device 103. The deviation device 101 is utilized for measuring the movement of users, for example flexion, extension, right lateral bending, left lateral bending, clockwise axial rotation, anticlockwise axial rotation, clockwise circumduction, and anticlockwise circumduction. Suitable devices can include BTE Work Stimulator II™, goniometers, Isotechnologies Back Tracker™, Assension Motionstar™, Peak 5™ Motus, or postural video analysis. From the movements, measurements may be made, including but not limited to extension, flexion, lateral bend and axial rotation range, mean and peak velocity, and clockwise and counter-clockwise circumduction area.

The display device 103 is utilized to train the user 104 when utilizing the deviation device 101. Training occurs by a target (not shown) being moved around the display 103 and requiring the user 104 to move in tandem with the target. The display 103 can be a fish tank display, cave automatic virtual environment, or a head mounted display. In one embodiment, the display 103 is attached to a computer 105 having a processor, memory such as RAM or ROM, power supply, and user interface such as keyboard, mouse, microphone, and speaker. The computer 105 preferably has stored thereon algorithms for directing the target movement, and receiving and storing movement measurements of the user 104. The computer 105 also delivers feedback to the user 104 based on the received measurements, such as visual, auditory, or success rate feedback. Information may be communicated back and forth from the deviation device 101 to the display device 103 and vice versa via wired or wireless means.

In a still further embodiment, other implements may be utilized in the system 100, such as feedback gloves, to allow a more fuller body utilization by the user 104.

FIG. 2 shows the system 200 of the present invention from a side view, wherein a user 202 moves in response to a target icon on a display device 203. The user's 202 movement is reassured by the use of a deviation device 201.

FIG. 3 is an embodiment of the system of the present invention. Alike the previous embodiment, the system include a deviation device 301, a computer 305, and sensors 307, however, this embodiment possesses a head movement display (HMD) 303 as a display. HMD are well-known in the art, for example the HMDs taught in U.S. Pat. Nos. 4,968,123, 5,130,794, and 5,436,765, incorporated herein by reference, are suitable for use.

The present invention also includes a method for rehabilitation of low back pain patients utilizing the present system.

In a first step, the trunk of a user is measured via sensors 401, such as cameras. A target icon is then displayed on the display 403. In one embodiment, the target icon can be merely a cursor, i.e., a movable spot of light, on the display. In other embodiments, the target icon can be designed like a real world object such as a ball, for example a tennis ball, or a golf ball.

The target icon is then moved or manipulated 405 on the display. In one embodiment, the icon is randomly moved throughout the display. In another embodiment, the target icon instructs the user by positioning itself so the user can simulate hitting or striking the icon. Following, the user should emulate or copy the movement of the target icon 407. As stated previously, the user will be required to move in a variety of ways. The user's movement will be tracked via the sensors focused on the user. Data from the sensors will be forwarded to the computer for analysis.

The results of the user's movement when compared to the target icon will be displayed 409 on the display, thus providing feedback to the user. Through the displayed results, the user will be able to gauge his movement. Depending on the user's performance, the movement of the target icon will increase to difficulty 411.

In another embodiment, the results are displayed to both a user and a therapist. Further, the results may be forwarded over data lines to persons located a distance from the user.

### EXAMPLE 1

For measuring a user's movement, a BackTracker™ system was used. Participants were asked to carry out a series of movements at a comfortable speed and in a comfortable method. The set of movement was repeated twice on each testing day. The movement of the user's was recorded via sensors on the computer, with results being displayed to the user and therapist.

FIG. 5 is an embodiment of the feedback results displayed to the user.

Having described embodiments of the present system with reference to the accompanying drawings, it is to be understood that the present system is not limited to the precise embodiments, and that various changes and modifications may be effected therein by one having ordinary skill in the art without departing from the scope or spirit as defined in the appended claims.

In interpreting the appended claims, it should be understood that:

a) the word “comprising” does not exclude the presence of other elements or acts than those listed in the given claim;

b) the word “a” or “an” preceding an element does not exclude the presence of a plurality of such elements;

c) any reference signs in the claims do not limit their scope;

d) any of the disclosed devices or portions thereof may be combined together or separated into further portions unless specifically stated otherwise; and

e) no specific sequence of acts or steps is intended to be required unless specifically indicated.

1. A system for measuring back movement of a chronic low back pain patient, comprising:
a posture and joint deviation device;
one or more sensors;
a display;
wherein said display possesses algorithms for training a user and algorithms for providing feedback to said user.

2. The system for measuring back movement of a chronic low pain patient of claim 1, wherein said posture and joint deviation device is a goniometer or postural video analysis.

3. The system for measuring back movement of a chronic low pain patient of claim 2, wherein said posture and joint deviation device is capable of measuring one or more from the group consisting of extension, flexion, lateral bend, axial rotation range, mean velocity, peak velocity, clockwise circumduction, and counter-clockwise circumduction.

4. The system for measuring back movement of a chronic low pain patient of claim 1, wherein said sensors are cameras.

5. The system for measuring back movement of a chronic low pain patient of claim 1, wherein said display is a fish tank display, cave automatic virtual environment, or a head mounted display.

6. The system for measuring back movement of a chronic low pain patient of claim 1, further comprising a computer.

7. The system for measuring back movement of a chronic low pain patient of claim 1, further comprising feedback gloves.

8. A method for measuring the back movement of a chronic low back patient, comprising the steps of:
measuring the trunk orientation of said patient;
displaying a target icon on a display;
moving said target icon on said display;
emulating, by said patient, the movement of said target icon;
displaying results of patient's emulation of said target icon; and
increasing in difficulty the movement of said target icon.

9. The method for measuring the back movement of a chronic low back patient of claim 8, whereby measuring the trunk orientation occurs via camera sensors.

10. The method for measuring the back movement of a chronic low back patient of claim 8, whereby displaying said target icon occurs on a display selected from the group consisting of fish tank display, head mounted display, and cave automatic virtual environment.

11. The method for measuring the back movement of a chronic low back patient of claim 8, moving said target icon occurs via algorithms stored on a computer.

12. The method for measuring the back movement of a chronic low back patient of claim 8, whereby emulation by the patient occurs via one or more movements selected from the group consisting of flexion, extension, right lateral bending, left lateral bending, clockwise axial rotation, anti-clockwise axial rotation, clockwise circumduction, and anti-clockwise circumduction.

13. The method for measuring the back movement of a chronic low back patient of claim 8, wherein displaying results occurs via feedback displayed to said patient via said display.

14. The method for measuring the back movement of a chronic low back patient of claim 8, wherein increasing difficulty in the movement of said target icon occurs by increasing the speed of the movement of the target icon, or increasing the complexity of the movement of the target icon.
